(19) United States
(12) Patent Application Publication
(10) Pub. No.: US 2025/0002970 A1
(43) Pub. Date: Jan. 2, 2025

(54) AMPLIFIABLE METABOLIC LABELING FOR DETECTION OF BACTERIAL VIABILITY, GROWTH AND ANTIBIOTIC SUSCEPTIBILITY

(71) Applicant: University of Massachusetts, Boston, MA (US)

(72) Inventors: Emily S. Melzer, San Francisco, CA (US); Carolina Santamaria, Sunderland, MA (US); Eugenia Roberts, Cambridge, MA (US); Thameez M. Koyasseril-Yehiya, Amherst, MA (US); M. Sloan Siergrist, Northampton, MA (US)

(21) Appl. No.: 18/688,729

(22) PCT Filed: Sep. 2, 2022
(86) PCT No.: PCT/US2022/075885

§ 371 Date: Mar. 1, 2024

Related U.S. Application Data
(60) Provisional application No. 63/260,897, filed on Sep. 3, 2021.

(51) Int. Cl. C12Q 1/18; C12R 1/19; C12R 1/34; C12R 1/445

(52) U.S. Cl. CPC C12Q 1/18; C12R 2001/19; C12R 2001/34; C12R 2001/445

(57) ABSTRACT

Provided herein is a rapid amplifiable readout of bacterial growth, such as an ELISA paired with metabolic labeling. Methods comprise a) contacting a sample from a subject suffering from a bacterial infection with an antibiotic; and b) determining the effect, if any, of the antibiotic on incorporation of a single- or di-D-amino acid into peptidoglycan of the bacterial cell or incorporation of trehalose into the bacterial cell envelope, wherein an antibiotic that reduces, as compared to control without the antibiotic being present, incorporation of the D-amino acid into peptidoglycan of the bacterial cell or inhibited incorporation of trehalose into the bacterial cell envelope is an antibiotic that is effective.

### PRIORITY APPLICATION(S)

This application claims priority to U.S. Provisional Patent Application Ser. No. 63/260,897, filed on Sep. 3, 2021, the disclosure of which is incorporated by reference herein in its entirety.

### GOVERNMENT GRANT SUPPORT

This invention was made with government support under DP2 AI138238 awarded by the National Institutes of Health. The government has certain rights in the invention.

### BACKGROUND OF THE INVENTION

Bacterial infections, such as bloodstream bacterial infections, are a significant source of morbidity and mortality in the U.S. Patient outcome is dependent on the time it takes to begin appropriate antibiotic treatment. Current hospital protocols rely on culture-based diagnosis, a multi-step, approximately three-day process.

Bacterial contamination in an industrial setting, such as a food production facility or biomedical reagent facility, is also an issue, resulting in use a days-long cultivation process to check for bacterial contaminants.

### SUMMARY OF INVENTION

Herein, is provided a rapid amplifiable readout of bacterial growth, such as an ELISA paired with metabolic labeling. This assay dramatically shortens the time to drug susceptibility profiling of pathogenic bacteria, including blood-borne bacteria, to detect metabolic labeling of cell wall peptidoglycan in live Gram-positive and Gram-negative bacteria or other layers of the cell envelope, such as those containing trehalose. The invention can be used as a patient diagnostic too, as well as a tool to detect bacterial growth in an industrial setting and/or hospital facility setting (buildings, rooms, beds, beds, chairs etc.), such as a food production facility, biomedical reagent or device facility, non-food products facility and products, such as a home care products (cleaning products), personal care products (cosmetics (skin care, hair care, make up, baby care products; and toiletries (toothcare, feminine care products))), chemical products and toys, including, but not limited to, tattoo ink and children's toys such as soap bubbles, play dough and paints, as well water for drinking or other uses.

Metabolic labeling (of cell wall peptidoglycan or of other biomolecules, such as trehalose) is generally detected by radioactivity, fluorescence and/or immunoblot. While these methods work well in a research laboratory setting, they are not necessarily well-suited to clinical applications. Provided herein is an ELISA paired with metabolic labeling for use as a diagnostic, such as a clinical diagnostic. This diagnostic provides a rapid, amplifiable readout for active bacterial growth.

One embodiment provides a method to determine if an antibiotic is effective to treat a bacterial infection in a subject comprising: a) contacting a sample from a subject suffering from a bacterial infection with an antibiotic; and b) determining the effect, if any, of the antibiotic on incorporation of a single- or di-D-amino acid into peptidoglycan of the bacterial cell or incorporation of trehalose into the bacterial cell envelope, wherein an antibiotic that reduces, as compared to control without the antibiotic being present, incorporation of the D-amino acid into peptidoglycan of the bacterial cell or inhibited incorporation of trehalose into the bacterial cell envelope is an antibiotic that is effective to treat said bacterial infection in said subject.

Another embodiment provides a method to treat a subject for a bacterial infection comprising: a) contacting a sample from a subject suffering from a bacterial infection with an antibiotic; b) determining the effect of the antibiotic on incorporation of a single- or di-D-amino acid into peptidoglycan of the bacterial cell or trehalose incorporation; and c) administering the antibiotic to said subject, wherein said antibiotic inhibited incorporation of the D-amino acid into peptidoglycan of the bacterial cell or inhibited incorporation of trehalose into the bacterial cell envelope, so as to treat said subject for a bacterial infection. Another embodiment provides a method to detect antibiotic resistant strains of bacteria in a subject comprising: a) contacting a sample from a subject suffering from a bacterial infection with an antibiotic; and b) determining the effect, if any, of the antibiotic on incorporation of a single- or di-D-amino acid into peptidoglycan of the bacterial cell or trehalose incorporation, wherein the bacteria is resistant to said antibiotic if said bacteria continue to grow and incorporate the D-amino acid into peptidoglycan of the bacterial cell or trehalose into the bacterial cell envelope.

One embodiment provides a method to detect bacterial contamination of a site comprising, a) contacting a sample from a site with an antibiotic; and b) determining the effect, if any, of the antibiotic on incorporation of a single- or di-D-amino acid into peptidoglycan of the bacterial cell or trehalose incorporation, wherein an antibiotic that reduces, as compared to control without the antibiotic being present, incorporation of the D-amino acid into peptidoglycan of the bacterial cell or inhibited incorporation or trehalose into the bacterial cell envelope is indicative of bacterial contamination of said site. In one embodiment, the site is an industrial setting and/or hospital facility setting (buildings, rooms, beds, beds, chairs etc.), including a food production facility (factory or bakery), biomedical reagent or device facility, non-food products facility and products, such as a home care products (cleaning products), personal care products (cosmetics (skin care, hair care, make up, baby care products; and toiletries (toothcare, feminine care products))), chemical products and toys, including, but not limited to, tattoo ink and children's toys such as soap bubbles, play dough and paints, as well water for drinking or other uses. In another embodiment, the contaminated site is decontaminated (through the use of one or more antibiotics and/or one or more cleaning products (such as bleach) and optionally progress of said decontamination is monitored by repeating steps a) and b) one or more times.

In one embodiment, the sample comprises cells and the bacteria are present in intracellularly in said cells. In another embodiment, the bacterial infection is caused by a pathogenic bacterium, such as one or more of the bacteria that contribute to sepsis. In one embodiment the bacterium is a mycobacterium.

In one embodiment, the D-amino acid is modified with a bioorthogonal functional group, including an azide, an alkyne, or a norbornene group. In one embodiment, the D-amino acid and/or trehalose is labeled with a detectable label, including biotin, a radioactive isotope, a fluorescent label or a peptide that can be detected by antibody binding. In one embodiment, the probe is detected with an antibody.

In one embodiment, the identity of the bacteria causing the infection is not determined.

In one embodiment, b) is carried out by an ELISA assay. In one embodiment, b) and c) are carried out by a lateral flow assay.

### BRIEF DESCRIPTION OF THE DRAWINGS

FIG. 1. Metabolic labeling. Top, an envelope precursor (green) with reactive group X is incorporated into the bacterial surface (grey) by enzymatic machinery. The presence of the probe is revealed by reaction with a label bearing complementary reactive group Y (or by antibody). Bottom, in some cases the probe can be directly functionalized. Yellow star, detection moiety such as a fluorophore or biotin.

FIG. 2. Surface labeling of bacteria+/−antibiotics. Mycobacterium smegmatis was transformed or not with a plasmid bearing a resistance cassette for either kanamycin\ (kanR) or hygromycin (hygR). Bacteria were incubated+/−drug for ˜2.5 doublings (7.5 hours) then labeled for 30 min with a cell surface probe followed by ligation to a fluorophore. MFI, median fluorescence intensity from flow cytometry quantitation. DCS, D-cycloserine, amp, ampicillin, imi, imipenem, INH, isoniazid, rif, rifampicin.

FIGS. 3A-3D. Growth dependent cell surface probe incorporates rapidly into E. coli and S. aureus growing cells. S. aureus (a,b) and E. coli (c,d) were labeled with probe followed by ligation to fluorophore (or can be by antibody only). Cells were imaged using fluorescence microscopy (a,c). Fluorescence intensity was measured by flow cytometry (b,d).

FIG. 4. ELISA can be used as a rapid readout for antibiotic susceptibility. S. aureus was incubated+/−antibiotic for 20 min then with cell envelope probe for an additional 10 min. Probe incorporation was quantitated by sandwich ELISA (absorbance, OD₄₅₀).

FIGS. 5-7 provides lateral flow assay embodiments.

### DETAILED DESCRIPTION OF THE INVENTION

Bloodstream bacterial infections are a significant source of morbidity and mortality in the U.S. Patient outcome is dependent on the time it takes to begin appropriate antibiotic treatment. Current hospital protocols rely on culture-based diagnosis, a multi-step, approximately 3-day process. A labeling method was developed to rapidly, selectively and covalently tag the cell surface of actively growing bacteria. This labeling is modular, allowing one to detect cell surface-embedded probes with labels such as fluorophores and affinity handles. As provided herein, this labeling can provide a rapid amplifiable readout of bacterial growth that will dramatically shorten the time to drug susceptibility profiling of blood-borne pathogenic bacteria. It is also time consuming to detect bacterial growth in non-patient settings, such as industrial settings where the instant invention will provide such facilities with a rapid readout to detect bacterial contaminants at very small cell numbers.

### Definitions

In describing and claiming the invention, the following terminology will be used in accordance with the definitions set forth below. Unless defined otherwise, all technical and scientific terms used herein have the same meaning as commonly understood by one of ordinary skill in the art to which this invention belongs. Any methods and materials similar or equivalent to those described herein can be used in the practice or testing of the present invention. Specific and preferred values listed below for radicals, substituents, and ranges are for illustration only; they do not exclude other defined values or other values within defined ranges for the radicals and substituents.

As used herein, the articles “a” and “an” refer to one or to more than one, i.e., to at least one, of the grammatical object of the article. By way of example, “an element” means one element or more than one element.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

As used herein the term “isolated” is meant to describe a compound, cell or bacterium of interest that is in an environment different from that in which the compound naturally occurs. “Isolated” is meant to include compounds, cells or bacteria that are within samples that are substantially enriched for the compound, cell or bacterium of interest and/or in which the compound, cell or bacterium of interest is partially or substantially purified.

As used herein, the term “substantially purified” refers to a compound, cell or bacterium that is removed from its natural environment or its synthetic environment and is at least 60% free, at least 75% free, at least 90% free, at least 95% free, at least 98% free, or at least 99% free from other components, cell or bacterium with which it is naturally associated, or is at least 60% free, at least 75% free, at least 90% free, at least 95% free, at least 98% free, or at least 99% free from contaminants associated with synthesis of the compound, cell or bacterium.

“Optional” or “optionally” means that the subsequently described event, circumstance, feature, or element may, but need not, occur, and that the description includes instances where the event or circumstance occurs and instances in which it does not.

As used herein, a disease, disorder or condition (such as a bacterial infection) is “treated” if at least one pathophysiological measurement associated with the disease is decreased and/or progression of a pathophysiological process is reversed, halted or reduced. In another example, a disease, disorder or condition can be “treated” if one or more symptoms of the disease or disorder is reduced, alleviated, terminated, slowed, or prevented. Measurement of one or more exemplary clinical hallmarks and/or symptoms of a disease can be used to aid in determining the disease status in an individual and the treatment of one or more symptoms associated therewith.

The term “administering” as used herein refers to administering to a subject. This includes administration an antibiotic to a subject which is suffering from a bacterial infection. Administration “in combination with” one or more further agents include concurrent and consecutive administration, in any order.

The term “subject” includes eukaryotes, including mammals, such as humans, higher non-human primates, rodents, and domestic and farm animals, such as cows, horses, dogs, and cats. In some embodiments of the invention, the mammal is a human.

The term “pharmaceutical composition” refers to a formulation containing the disclosed compounds in a form suitable for administration to a subject. In one embodiment, the pharmaceutical composition is in bulk or in unit dosage form. The unit dosage form is any of a variety of forms, including, for example, a tablet, capsule, or a vial. The quantity of active ingredient in a unit dose of composition is an effective amount and is varied according to the particular treatment involved.

The phrase “therapeutically effective amount” or “effective amount” used in reference to an agent of the invention is an art-recognized term. In certain embodiments, the term refers to an amount of an agent that produces some desired effect at a reasonable benefit/risk ratio applicable to any medical treatment. In certain embodiments, the term refers to that amount necessary or sufficient to eliminate, reduce or maintain a target of a particular therapeutic regimen. The effective amount may vary depending on such factors as the disease or condition being treated, the particular targeted constructs being administered, the size of the subject or the severity of the disease or condition. One of ordinary skill in the art may empirically determine the effective amount of a particular compound without necessitating undue experimentation.

“Inhibitors,” “activators,” and “modulators” are used to refer to activating, inhibitory, or modulating (increase, inhibit, decrease or activate expression or activity as compared to control (an untreated or healthy subject/mammal and/or cell) molecules.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

The terms “comprises,” “comprising,” and the like can have the meaning ascribed to them in U.S. Patent Law and can mean “includes,” “including” and the like. As used herein, “including” or “includes” or the like means including, without limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

### Aspects of the Invention

Bacterial surface labelling is sensitive to growth inhibition by antibiotics, without ever having to first determine the specific type of bacteria. To determine which antibiotic(s) are appropriate to treat, for example, sepsis, a sample, such as a sample taken from a patient suffering from sepsis, is contacted with one or more antibiotics, then the effect of one or more antibiotics on the incorporation of a D-amino acid into bacterial cell peptidoglycan or incorporation of trehalose into the bacterial cell envelope is determined.

Whether one or more antibiotics reduces incorporation of a-modified D-amino acid into peptidoglycan (PG) present in a bacterial cell can be determined by contacting the bacterium with a reagent, as described herein, which reacts with a molecule or functional group conjugated to the D-amino acid present in the PG, where the reagent comprises a detectable label or an epitope to be recognized by an antibody. In some cases, the detectable label is a fluorescent label. Where one or more antibiotics inhibits growth, which in turn inhibits incorporation of a D-amino acid into new PG synthesized by a bacterial cell, the amount of label in the PG is reduced. A reduction in the amount of labeling indicates the one or more antibiotics that will be useful/appropriate to treat, for example, said sepsis or to clean an industrial setting and then follow up with post treatment or post decontamination testing to monitor progress. Following this determination, said sepsis can be treated or facility (if testing of a site was done) can be cleaned/decontaminated and product can be recalled as needed.

In another aspect, trehalose is used in place of, or in addition to, D-amino acid.

### Bacteria/Diseases

Gram-positive and Gram-negative bacteria (this classification system is based on a chemical staining procedure that reports the presence (Gram-negative) or absence (Gram-positive) of an outer membrane; the outer membrane is a permeability barrier that contributes to the intrinsic antibiotic susceptibility of a bacterium), including pathogenic and non-pathogenic species, can be screened for antibiotic susceptibility and/or treatment in the present invention.

Bacteria that can be modified such that PG present in the bacterial cell wall being synthesized comprises at least one D-amino acid include, but are not limited to, pathogenic intracellular bacteria (i.e., pathogenic bacteria that replicate in a host cell); pathogenic bacteria that do not replicate in a host cell; and non-pathogenic bacteria (e.g., non-pathogenic laboratory strains of bacteria). Non-pathogenic bacteria include commensal bacteria (present in a host) as well as free-living bacteria (living outside a host).

In some cases, the bacterium is an obligate intracellular pathogen or a facultative intracellular pathogen. Examples of such bacteria include, e.g., a Mycobacterium species. Examples of species of Mycobacterium include, but are not limited to, M. tuberculosis, M. bovis, M. bovis strain Bacillus calmette-guerin (BCG) including BCG substrains, M. avium, M. intracellulare, M. africanunum, M. kansasii, M. marinum, M. ulcerans and M. paratuberculosis. Examples of other obligate and facultative intracellular bacterial species include, but are not limited to, Legionella pneumophila, other Legionella species, Salmonella typhi, other Salmonella species, Shigella species, Listeria monocytogenes, Staphylococcus aureus, Staphylococcus epidermidis, Bacteroides fragilis, other Bacteroides species, Chlamydia pneumoniae, Chlamydia trachomatis, Chlamydia psittaci, Coxiella burnetii, other Rickettsial species, and Ehrlichia species.

Suitable bacteria include, but are not limited to, Francisella tularensis; Listeria monocytogenes; Salmonella; Brucella; Legionella pneumophila; Mycobacterium (e.g., M. tuberculosis, M. leprae, M. bovis, M. avium, M. abscessus); Nocardia (e.g., N. asteroids, N. farcinica, N. nova, N. transvalensis, N. brasiliensis, N. pseudobrasiliensis); Rhodococcus equui; Yersinia pestis; Neisseria (e.g., N. meningitidis, N. gonorrhoeae); Shigella (e.g., S. dysenteriae, S. flexneri, S. boydii, and S. sonnei); Chlamydia ((C. trachomatis, C. pneumoniae, C. psittaci); Rickettsia; and Coxsiella. Other suitable bacteria include, e.g., pathogens such as Vibrio cholerae, Pseudomonas aeruginosa, and pathogenic Escherichia coli; model organisms such as Escherichia coli, Bacillus subtilis, and Caulobacter cresentus; facultative pathogens such as Streptococcal and Clostridial species; and commensals such as Bacteroides thetaiotamicron. Or any other bacteria involved in sepsis or blood-borne bacterial pathogens.

Gram-positives including Staphylococcus aureus, Staphylococcus epidermidis, Bacillus cereus, Enterococcus faecalis, Enterococcus faecium, Listeria monocytogenes, Streptococcus pneumoniae, Streptococcus pyogenes and Streptococcus agalactiae and b) Gram-negatives including Escherichia coli, Klebsiella pneumoniae, Haemophilus influenzae, Pseudomonas aeruginosa, Acinetobacter baumannii, Neisseria meningitidis, Bacteroides fragilis and Proteus mirabilis.

### Sepsis

Sepsis is a life-threatening condition that arises when the body's response to infection causes injury to its own tissues and organs. This initial stage is followed by suppression of the immune system. Common signs and symptoms include fever, increased heart rate, increased breathing rate, and confusion. There may also be symptoms related to a specific infection, such as a cough with pneumonia, or painful urination with a kidney infection. The very young, old, and people with a weakened immune system may have no symptoms of a specific infection, and the body temperature may be low or normal instead of having a fever. Severe sepsis causes poor organ function or blood flow. The presence of low blood pressure, high blood lactate, or low urine output may suggest poor blood flow. Septic shock is low blood pressure due to sepsis that does not improve after fluid replacement.

Sepsis is caused by many organisms including bacteria, viruses and fungi. Common locations for the primary infection include the lungs, brain, urinary tract, skin, and abdominal organs. Risk factors include being very young, older age, a weakened immune system from conditions such as cancer or diabetes, major trauma, or burns.

Sepsis requires immediate treatment with intravenous fluids and antimicrobials (including but not limited to, vancomycin, teicoplanin, macrolide, fluoroquinolone, colistin, beta-lactam antibiotics, penicillin, amoxicillin, doxycycline, cephalexin, ciprofloxacin, clindamycin, metronidazole, azithromycin, sulfamethoxazole, trimethoprim, Amoxicillin, Ampicillin, Penicillin G, Penicillin V, cephalosporins, Cefixime, Cefpodoxime, Cefuroxime, Cephalexin, macrolides, Azithromycin (‘Z-pak’), Clarithromycin, Erythromycin, Fluoroquinolones (or quinolones), Ciprofloxacin, Levofloxacin, Moxifloxacin, sulfonamides, Sulfacetamide, Sulfadiazine, Sulfamethoxazole-Trimethoprim, tetracycline, Doxycycline, Minocycline, Tetracycline, clindamycin, metronidazole and nitrofurantoin), including other treatments (fluids, intravenous fluids, blood products, vasopressors, steroids, and/or anesthesia). Antimicrobials such as broad-spectrum antibiotics (one or more β-lactam antibiotic with broad coverage, antibiotics specific to gram-negative organisms, or broad-spectrum carbapenem combined with fluoroquinolones, macrolides, or aminoglycosides). The choice of antibiotics is important in determining the survival of the person.

Antibiotic regimens can be assessed daily and altered if appropriate.

Disease severity partly determines the outcome. The risk of death from sepsis is as high as 30%, while for severe sepsis it is as high as 50%, and septic shock 80%. Sepsis affected about 49 million people in 2017, with 11 million deaths (1 in 5 deaths worldwide). In the developed world, approximately 0.2 to 3 people per 1000 are affected by sepsis yearly, resulting in about a million cases per year in the United States. Rates of disease have been increasing.

### Non-Patient Uses

The invention can be used as a tool to detect bacterial growth in an industrial setting and/or hospital facility setting (buildings, rooms, beds, beds, chairs etc.), such as a food production facility, biomedical reagent or device facility, non-food products facility and products, such as a home care products (cleaning products), personal care products (cosmetics (skin care, hair care, make up, baby care products; and toiletries (toothcare, feminine care products)), chemical products and toys, including, but not limited to, tattoo ink and children's toys such as soap bubbles, play dough and paints, as well water for drinking or other uses.

### Labelling/D-Amino Acids to Label Peptidoglycan

Bloodstream bacterial infections occur in 1.5 million U.S. patients per year. Standard, culture-based diagnosis is a multi-step, 3-day process that includes species identification. The best correlate of patient survival is the time elapsed prior to initiation of correct antibiotic treatment. Previously, a D-amino acid-based labeling method was developed that selectively and covalently tags the cell walls of actively growing bacteria. D-amino acids metabolically incorporate within minutes and can be revealed by a chemical reaction. This strategy provides an amplifiable readout of bacterial growth that will dramatically shorten drug susceptibility profiling and also does not require bacterial species classification.

D-amino acid-based labeling for the bacterial cell wall is disclosed in U.S. Pat. No. 9,303,068; Siegrist et al., ACS Chemical Biology 2013; Siegrist et al., FEMS Micro Rev 2015; https://www.ncbi.nlm.nih.gov/pmc/articles/PMC3589519/; U.S. patent application Ser. No. 16/048,024; and U.S. patent application Ser. No. 16/048,000, (which are incorporated herein by reference in their entireties). Peptidoglycan (PG) is a component of the bacterial cell wall. Peptidoglycan (PG) is a polymer comprising sugars and amino acids. The sugar component consists of alternating residues of β-(1,4) linked N-acetylglucosamine (GlcNAc) and N-acetylmuramic acid (MurNAc). Attached to the MurNAc is a peptide chain of three to five amino acids.

Modified D-amino acids allow labeling of peptidoglycan. The D-amino acid can be directly labeled with a detectable agent, such as biotin, or through the use of a linker. Further, any convenient bioorthogonal functional groups may be utilized in the modified D-amino acids and modified peptidoglycan. Bioorthogonal functional groups of interest include, but are not limited to azide, alkyne, alkene, norbornene, trans-cyclooctene and tetrazole. In some cases, the modified D-amino acids are azide-, alkyne-, or norbornene-modified D-amino acids.

D-amino acids that can be modified to produce a modified D-amino acid including any genetically encoded or non-encoded amino acid. In some embodiments, a modified D-amino acid is any one of the twenty encoded amino acids. In some embodiments, a modified D-amino acid, such as an azide-modified, norbornene-modified or alkyne-modified D-amino acid, suitable for incorporation into PG to generate a modified PG is a non-canonical D-amino acid. In some embodiments, a modified D-amino acid, such as an azide-modified, norbornene-modified or alkyne-modified D-amino acid, suitable for incorporation into PG to generate a modified PG is D-alanine. Diverse bacterial phyla produce and incorporate D-amino acids other than D-alanine into PG. See, e.g., Lam et al. (2009) Science 325:1552; and Cava et al. (2011) EMBO J. 30:3442. Thus, in some embodiments, a modified D-amino acid suitable for incorporation into PG to generate a modified PG is derived from D-methionine, D-leucine, D-tyrosine, D-phenylalanine, D-cysteine, or D-threonine, or D-isoleucine or a D-lysine. In some cases, a modified PG can comprise an azide-modified, a norbornene-modified or an alkyne-modified D-amino acid, where the modified amino acid is a non-encoded amino acid.

Any convenient linkers may be utilized in the subject modified D-amino acids.

In some cases, a subject modified PG comprises one or more naturally occurring non-genetically encoded L-amino acids, synthetic L-amino acids or D-enantiomers of an amino acid. A “non-naturally encoded amino acid” refers to an amino acid that is not one of the 20 common amino acids or pyrrolysine or selenocysteine. Other terms that may be used synonymously with the term “non-naturally encoded amino acid” are “non-natural amino acid,” “unnatural amino acid,” “non-naturally-occurring amino acid,” and variously hyphenated and non-hyphenated versions thereof.

### Labels/Reagents

The D-amino acid may be conjugated to any convenient reagent, such as a reagent that includes a detectable label. The reagent may include a compatible functional group that is capable of conjugating to the modified PG, e.g., via bioorthogonal conjugation with the bioorthogonal functional group (e.g., an azide, a norbornene or alkyne group) of a modified PG. A variety of bioorthogonal chemistries and reagents may be utilized in the subject modified PGs, D-amino acids and conjugation reagents, including but not limited to, Click chemistry groups and reagents (e.g., as described by Sharpless et al., (2001), “Click Chemistry: Diverse Chemical Function from a Few Good Reactions”, Angewandte Chemie International Edition 40 (11): 2004-2021), photoinduced cycloaddition chemistry groups and reagents (e.g., photo-click chemistry and groups, as described by Lin et al., “Photoinducible Bioorthogonal Chemistry: A Spatiotemporally Controllable Tool to Visualize and Perturb Proteins in Live Cells”, ACCOUNTS OF CHEMICAL RESEARCH 828-839′ 2011 Vol. 44, No. 9); norbornene-tetrazine chemistry groups and reagents (e.g., as described by Carell et al. “A Genetically Encoded Norbornene Amino Acid for the Mild and Selective Modification of Proteins in a Copper-Free Click Reaction”, Angew. Chem. Int. Ed. 1012, 51, 4466-4469); Staudinger ligation groups and reagents (e.g., as described by Bertozzi et al., (2000), “Cell Surface Engineering by a Modified Staudinger Reaction”, Science 287 (5460): 2007) (e.g., using azido and phosphine groups), and other bioconjugation groups and reagents (e.g., as described by Hermanson, Bioconjugate Techniques, Second Edition, Academic Press, 2008). In certain embodiments, the reagent includes a compatible functional group selected from an azido, a phosphine (e.g., a triaryl phosphine or a trialkyl phosphine or mixtures thereof), a dithiol, an active ester, an alkynyl, an alkenyl, a tetrazine, a tetrazole, a hydrazoyl chloride, and a norbornenyl.

In certain embodiments, the D-amino acid includes a detectable label (e.g., a florescent label).

### Detectable Labels

Exemplary detectable labels include, but are not necessarily limited to, molecules (e.g., auto-fluorescent molecules, molecules that fluoresce upon contact with a reagent, etc.), radioactive labels (e.g., ¹¹¹In, ¹²⁵I, ¹³¹I, ²¹²B, ⁹⁰Y, ¹⁸⁶Rh, and the like); biotin (e.g., to be detected through reaction of biotin and avidin); fluorescent tags; imaging reagents (e.g., those described in U.S. Pat. Nos. 4,741,900 and 5,326,856), and the like. Detectable labels also include peptides or polypeptides that can be detected by antibody binding, e.g., by binding of a detectably labeled antibody or by detection of bound antibody through a sandwich-type assay. Also suitable for use are quantum dots (e.g., detectably labeled semiconductor nanocrystals, such as fluorescently labeled quantum dots, antibody-conjugated quantum dots, and the like). See, e.g., Dubertret et al. 2002 Science 298:759-1762; Chan et al. (1998) Science 281:2016-2018; U.S. Pat. No. 6,855,551; Bruchez et al. (1998) Science 281:2013-2016.

Suitable fluorescent molecules (fluorophores) include, but are not limited to, fluorescein isothiocyanate, succinimidyl esters of carboxyfluorescein, succinimidyl esters of fluorescein, 5-isomer of fluorescein dichlorotriazine, caged carboxyfluorescein-alanine-carboxamide, Oregon Green 488, Oregon Green 514; Lucifer Yellow, acridine Orange, rhodamine, tetramethylrhodamine, Texas Red, propidium iodide, JC-1 (5,5′,6,6′-tetrachloro-1,1′,3,3′-tetraethylbenzimidazoylcarbocyanine iodide), tetrabromorhodamine 123, rhodamine 6G, TMRM (tetramethylrhodamine-, methyl ester), TMRE (tetramethylrhodamine, ethyl ester), tetramethylrosamine, rhodamine B and 4-dimethylaminotetramethylrosamine, green fluorescent protein, blue-shifted green fluorescent protein, cyan-shifted green fluorescent protein, red-shifted green fluorescent protein, yellow-shifted green fluorescent protein, 4-acetamido-4′-isothiocyanatostilbene-2,2′disulfonic acid; acridine and derivatives: acridine, acridine isothiocyanate; 5-(2′-aminoethyl)aminonaphthalene-1-sulfonic acid (EDANS); 4-amino-N-[3-vinylsulfonyl)phenyl]naphth-alimide-3,5 disulfonate; N-(4-anilino-1-naphthyl) maleimide; anthranilamide; 4,4-difluoro-5-(2-thienyl)-4-bora-3a,4a diaza-5-indacene-3-propioni-c acid BODIPY; cascade blue; Brilliant Yellow; coumarin and derivatives: coumarin, 7-amino-4-methylcoumarin (AMC, Coumarin 120), 7-amino-4-trifluoromethylcoumarin (Coumarin 151); cyanine dyes; cyanosine; 4′,6-diaminidino-2-phenylindole (DAPI); 5′,5″-dibromopyrogallol-sulfonaphthalein (Bromopyrogallol Red); 7-diethylamino-3-(4′-isothiocyanatophenyl)-4-methylcoumarin; diethylenetriaamine pentaacetate; 4,4′-diisothiocyanatodihydro-stilbene-2-,2′-disulfonic acid; 4,4′-diisothiocyanatostilbene-2,2′-disulfonic acid; 5-(dimethylamino) naphthalene-1-sulfonyl chloride (DNS, dansylchloride); 4-dimethylaminophenylazophenyl-4′-isothiocyanate (DABITC); eosin and derivatives: eosin, eosin isothiocyanate, erythrosin and derivatives: erythrosin B, erythrosin, isothiocyanate; ethidium; fluorescein and derivatives: 5-carboxyfluorescein (FAM),5-(4,6-dichlorotriazin-2-yl)amino-1-fluorescein (DTAF), 2′, 7′dimethoxy-4′5′-dichloro-6-carboxyfluorescein (JOE), fluorescein, fluorescein isothiocyanate, QFITC, (XRITC); fluorescamine; IR144; IR1446; Malachite Green isothiocyanate; 4-methylumbelli-feroneortho cresolphthalein; nitrotyrosine; pararosaniline; Phenol Red; B-phycoerythrin; o-phthaldialdehyde; pyrene and derivatives: pyrene, pyrene butyrate, succinimidyl 1-pyrene; butyrate quantum dots; Reactive Red 4 (Cibacron. TM. Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivative of sulforhodamine 101 (Texas Red); N,N,N′,N′-tetramethyl-6-carboxyrhodamine (TAMRA); tetramethyl rhodamine; tetramethyl hodamine isothiocyanate (TRITC); riboflavin; 5-(2′-aminoethyl)aminonaphthalene-1-sulfonic acid (EDANS),4-(4′-dimethylaminophenylazo)benzoic acid (DABCYL), rosolic acid; CAL Fluor Orange 560; terbium chelate derivatives; Cy 3; Cy 5; Cy 5.5; Cy 7; IRD 700; IRD 800; La Jolla Blue; phthalo cyanine; and naphthalo cyanine, coumarins and related dyes, xanthene dyes such as rhodols, resorufins, bimanes, acridines, isoindoles, dansyl dyes, aminophthalic hydrazides such as luminol, and isoluminol derivatives, aminophthalimides, aminonaphthalimides, aminobenzofurans, aminoquinolines, dicyanohydroquinones, and fluorescent europium and terbium complexes; and the like. Fluorophores of interest are further described in WO 01/42505 and WO 01/86001.

Suitable fluorescent proteins and chromogenic proteins include, but are not limited to, a green fluorescent protein (GFP), including, but not limited to, a GFP derived from Aequoria victoria or a derivative thereof, e.g., a “humanized” derivative such as Enhanced GFP, which is available commercially, e.g., from Clontech, Inc.; a GFP from another species such as Renilla reniformis, Renilla mulleri, or Ptilosarcus guemyi, as described in, e.g., WO 99/49019 and Peelle et al. (2001) J. Protein Chem. 20:507-519; “humanized” recombinant GFP (hrGFP) (Stratagene); any of a variety of fluorescent and colored proteins from Anthozoan species, as described in, e.g., Matz et al. (1999) Nature Biotechnol. 17:969-973; and the like.

### ELISA

Enzyme-linked immunosorbent assay (ELISA) can be used to detect cell surface probe incorporation. While there are other fluorescence readouts, more sensitive detection methods would significantly enhance the clinical utility of the metabolic labeling strategy. To this end, ELISA for detecting labeled bacteria is provided. ELISAs are sensitive, specific, straightforward and are used in the clinic to detect markers of disease, both infectious (HIV, Lyme, Zika) and chronic (anemia, squamous cell carcinoma, celiac).

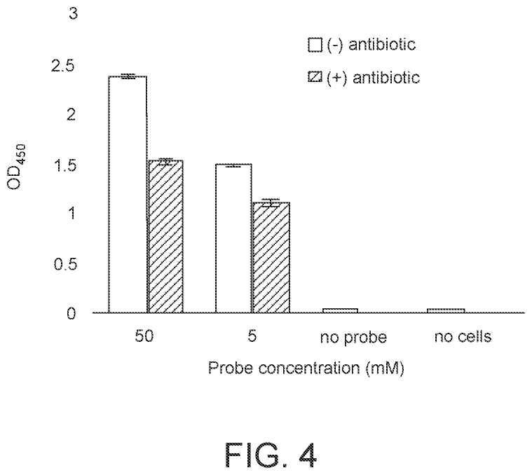

As an example, bacterial pathogens, such as blood borne pathogens, including but not limited to, E. coli and S. aureus labeled with, for example, a biotin-linked cell surface probe, (cell surface probes have been previously published (16)). The probe will incorporate efficiently into Gram-negatives and Gram-positives alike. The labeling can be quantified via a sandwich ELISA in which the wells are coated with streptavidin (a biotin binding partner), incubated with biotin-labeled bacteria (whole, gently permeabilized, or lysed) and detected with streptavidin conjugated to horseradish peroxidase (HRP) (FIG. 4). These experiments provide proof-of-concept for a novel, clinically relevant means of detecting bacterial growth via active cell envelope metabolism.

### Lateral Flow Assays (LFAs)

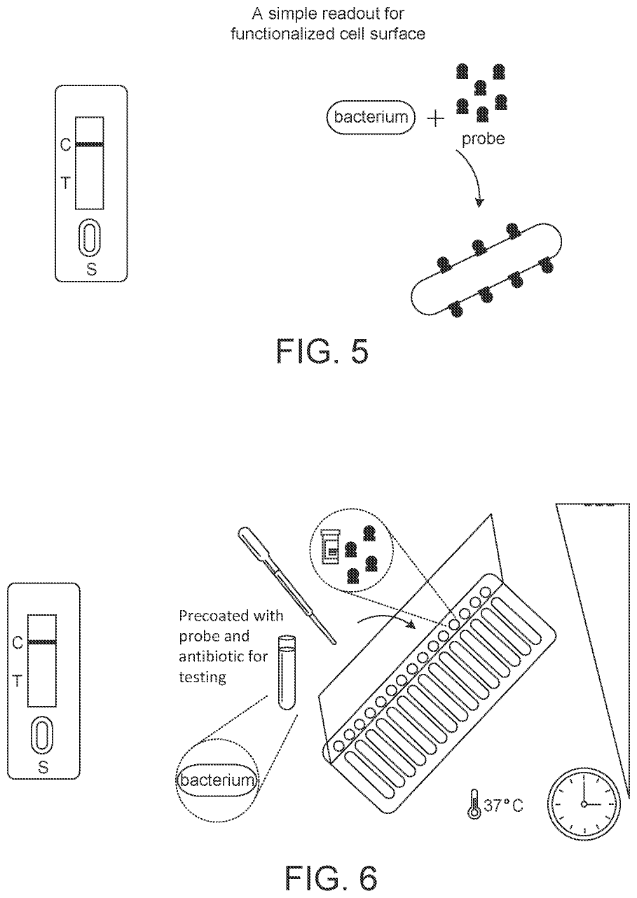

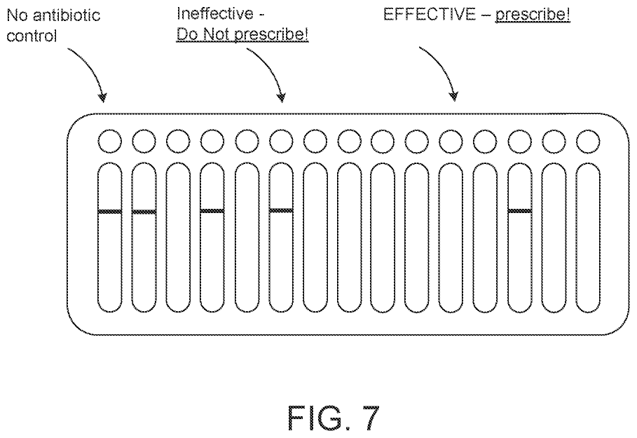

A lateral flow assay (LFA) is based on the movement of a liquid sample though a polymeric strip with attached molecules that interact with the analyte, providing a signal that can be visually detected (see, for example, FIGS. 5-7). LFA is generally a paper-based platform for the detection and/or quantification of analytes (such as proteins, haptens, nucleic acids and amplicons) in what are often complex mixtures, where the sample is placed on a test device and the results are displayed within about 5-30 min, such 5-10 minutes. Low development costs and ease of production of LFAs have resulted in the expansion of its applications to multiple fields in which rapid tests are needed, such as biomedicine, agriculture, food and environmental sciences. LFA-based tests are used in hospitals, physician's offices and clinical laboratories for the qualitative and quantitative detection of specific antigens and antibodies, as well as products of gene amplification, in such settings as veterinary medicine, quality control, product safety in food production, and environmental health and safety, including to screen for animal and human diseases, pathogens, chemicals, toxins and water pollutants, among others.

For LFA, a liquid sample (such as urine, saliva, sweat, serum, plasma, whole blood and other fluids (e.g., from a site)) containing the analyte of interest moves without the assistance of external forces (capillary action) through various zones of polymeric strips, on which molecules that can interact with the analyte are attached. A typical lateral flow test strip can consist of overlapping membranes that are mounted on a backing card for better stability. The sample is applied at one end of the strip, on the adsorbent sample pad, which can be loaded with buffer salts and surfactants that make the sample suitable for interaction with the detection system. The sample migrates through the conjugate release pad, which contains antibodies that are specific to the target analyte and are conjugated to colored or fluorescent particles-such as colloidal gold and latex microspheres (depending on the elements of recognition used, LFAs can be categorized into different types, such as ‘lateral flow immunoassays’ (LFIAs), in which antibodies are used as recognition elements, and nucleic acid LFA (NALFA), in which the detection of amplicons which can be formed during the polymerase chain reaction (PCR) are used). The sample, together with the conjugated antibody bound to the target analyte, migrates along the strip into the detection zone. This is generally a porous membrane (usually composed of nitrocellulose) with specific biological components (mostly antibodies or antigens) immobilized in lines. Their role is to react with the analyte bound to the conjugated antibody. Recognition of the sample analyte results in an appropriate response on the test line, while a response on the control line indicates the proper liquid flow through the strip. The read-out, represented by the lines appearing with different intensities, can be assessed by eye or using a dedicated reader (device).

Provided herein is a point-of-care multiple diagnostic assay with multiple test lines allowing the rapid and simultaneous detection of multiple analytes present in samples, providing a powerful tool for bacteria detection and progression, for example, before, after and/or during treatment. In order to test multiple analytes simultaneously under the same conditions, additional test lines of antibodies specific to different analytes can be immobilized in an array format. On the other hand, multiple test lines loaded with the same antibody can be used for semi-quantitative assays. The principle of this ‘ladder bars’ assay is based on the stepwise capture of colorimetric conjugate-antigen complexes by the immobilized antibody on each successive line, where the number of lines appearing on the strip is directly proportional to the concentration of the analyte. The liquid flows across the device because of the capillary force of the strip material and, to maintain this movement, an absorbent pad can be attached at the end of the strip. The role of the absorbent pad is to wick the excess reagents and prevent backflow of the liquid. A current example of an LFA is a pregnancy test stick.

Two formats of the LFIA can be distinguished: direct and competitive. A direct test is used for larger analytes such as the p24 antigen used in the human immunodeficiency virus (HIV) test as well as analytes with multiple antigenic sites such as human chorionic gonadotropin (hCG) used in pregnancy tests. The hCG test is an example of a sandwich-based assay, where the target is immobilized between two complementary antibodies. In the direct test, the presence of the test line indicates a positive result and the control line usually contains species-specific anti-immunoglobulin antibodies, specific for the antibody in the particular conjugate. In the case of small molecules with single antigenic determinants, which cannot bind to two antibodies simultaneously, competitive tests are used. In this type of test, the analyte blocks the binding sites on the antibodies on the test line, preventing their interactions with the colored conjugate. Therefore, a positive result is indicated by the lack of signal in the test line, while the control line should be visible independently of the test result.

As for a label colloidal gold is a widely used label in commercial LFIA. Another popular label is latex, which can be tagged with a variety of detector reagents such as colored or fluorescent dyes, and magnetic or paramagnetic components. As latex can be produced in multiple colors, it has an application in multiplex assays, which require discrimination between numerous lines. Carbon and fluorescent labels, or enzymatic modification of the labels, are also used. Carbon nanotubes, fluorescent labels, quantum dots, upconverting phosphors can all be used as labels. Another detection system that can be used is FACTT, an acronym for a sensitive protein detection system whereby amplification of the detection mAb occurs when coupled with T7 polymerase. Rather than measuring the mAb directly, the reader detects RNA molecules generated by the polymerase, thus greatly amplifying the result. This test can result in a qualitative color change but may also benefit from a reader (device). It may take 20-30 minutes.

There are many advantageous to using such an assay including, for example, point-of-care, providing inexpensive, rapid and easy tests desirable in many industries/countries and because of their long shelf life and the fact that refrigeration is often not required for storage, these tests are well suited for use in developing countries, small ambulatory care settings, remote regions and battlefields. Further as the visual result is usually clear, no additional equipment is needed; however, an optional device can be used for the readout.

### Treatment/Administration (Patient or Non-Patient Uses)

The antibiotics can be formulated and/or administered according to acceptable protocols. Suitable routes of administration include, e.g., parenteral and enteral routes of administration. Suitable routes of administration include, but are not limited to, intranasal, oral, mucosal, sublingual, transdermal, and transmucosal. An antibiotic can be administered via oral administration, rectal administration, parenteral, intravenous administration, intravitreal administration, intramuscular administration, inhalation, intranasal administration, topical administration, ophthalmic administration, or otic administration.

For example, antibiotics can be administered orally as discrete dosage forms, wherein such dosage forms include, but are not limited to, capsules, gelatin capsules, caplets, tablets, chewable tablets, powders, granules, syrups, flavored syrups, solutions or suspensions in aqueous or non-aqueous liquids, edible foams or whips, and oil-in water liquid emulsions or water-in-oil liquid emulsions.

The capsules, gelatin capsules, caplets, tablets, chewable tablets, powders or granules, used for the oral administration of the antibiotics compound are prepared by admixing a subject antibiotic (active ingredient) together with at least one excipient using conventional pharmaceutical compounding techniques. Non-limiting examples of excipients used in oral dosage forms described herein include, but are not limited to, binders, fillers, disintegrants, lubricants, absorbents, colorants, flavors, preservatives and sweeteners.

Non-limiting examples of suitable binders include, but are not limited to, corn starch, potato starch, starch paste, pre-gelatinized starch, or other starches, sugars, gelatin, natural and synthetic gums such as acacia, sodium alginate, alginic acid, other alginates, tragacanth, guar gum, cellulose and its derivatives (by way of example only, ethyl cellulose, cellulose acetate, carboxymethyl cellulose calcium, sodium carboxymethylcellulose, methyl cellulose, hydroxypropyl methylcellulose and microcrystalline cellulose), magnesium aluminum silicate, polyvinyl pyrrolidone and combinations thereof.

Non-limiting examples of suitable fillers include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. In certain embodiments, the binder or filler in pharmaceutical compositions provided herein are present in from about 50 to about 99 weight percent of the pharmaceutical composition or dosage form.

Non-limiting examples of suitable disintegrants include, but are not limited to, agar-agar, alginic acid, sodium alginate, calcium carbonate, sodium carbonate, microcrystalline cellulose, croscarmellose sodium, crospovidone, polacrilin potassium, sodium starch glycolate, potato or tapioca starch, pre-gelatinized starch, other starches, clays, other algins, other celluloses, gums, and combinations thereof. In certain embodiments, the amount of disintegrant used in the pharmaceutical compositions provided herein is from about 0.5 to about 15 weight percent of disintegrant, while in other embodiments the amount is from about 1 to about 5 weight percent of disintegrant.

Non-limiting examples of suitable lubricants include, but are not limited to, stearate, calcium stearate, magnesium stearate, stearic acid, mineral oil, light mineral oil, glycerin, sorbitol, mannitol, polyethylene glycol, other glycols, sodium lauryl sulfate, talc, hydrogenated vegetable oil (by way of example only, peanut oil, cottonseed oil, sunflower oil, sesame oil, olive oil, corn oil, and soybean oil), zinc stearate, sodium oleate, ethyl oleate, ethyl laureate, agar, silica, a syloid silica gel (AEROSIL 200, manufactured by W. R. Grace Co. of Baltimore, Md.), a coagulated aerosol of synthetic silica (marketed by Degussa Co. of Plano, Tex.), CABIO O-SIL (a pyrogenic silicon dioxide product sold by Cabot Co. of Boston, Mass.) and combinations thereof. In certain embodiments, the amount of lubricants used in the pharmaceutical compositions provided herein is in an amount of less than about 1 weight percent of the pharmaceutical compositions or dosage forms.

Non-limiting examples of suitable diluents include, but are not limited to, lactose, dextrose, sucrose, mannitol, sorbitol, cellulose, glycine or combinations thereof.

In certain embodiments, antibiotic is orally administered as a controlled release dosage form. Such dosage forms are used to provide slow or controlled release of an antibiotic. Controlled release is obtained using, for example, hydroxypropylmethyl cellulose, other polymer matrices, gels, permeable membranes, osmotic systems, multilayer coatings, microparticles, liposomes, microspheres, or a combination thereof.

Administration of antibiotics as oral fluids such as solution, syrups and elixirs are prepared in unit dosage forms such that a given quantity of solution, syrups or elixirs contains a predetermined amount of antibiotic. Syrups are prepared by dissolving the compound in a suitably flavored aqueous solution, while elixirs are prepared through the use of a non-toxic alcoholic vehicle. Suspensions are formulated by dispersing the compound in a non-toxic vehicle. Non-limiting examples of excipients used in as oral fluids for oral administration include, but are not limited to, solubilizers, emulsifiers, flavoring agents, preservatives, and coloring agents. Non-limiting examples of solubilizers and emulsifiers include, but are not limited to, water, glycols, oils, alcohols, ethoxylated isostearyl alcohols and polyoxy ethylene sorbitol ethers. Non-limiting examples of preservatives include, but are not limited to, sodium benzoate. Non-limiting examples of flavoring agents include, but are not limited to, peppermint oil or natural sweeteners or saccharin or other artificial sweeteners.

In certain embodiments, pharmaceutical compositions comprising an antibiotic are administered parenterally by various routes including, but not limited to, subcutaneous, intravenous (including bolus injection), intramuscular, and intraarterial.

Parenteral dosage forms are administered in the form of sterile or sterilizable solutions, suspensions, dry and/or lyophilized products ready to be dissolved or suspended in a pharmaceutically acceptable vehicle for injection (reconstitutable powders) and emulsions. Vehicles used in such dosage forms include, but are not limited to, Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Antibiotics can be any antibiotic available to the art, including, but not limited to, antimicrobials, including, but not limited to, vancomycin, teicoplanin, macrolide, fluoroquinolone, colistin, imipenem, isoniazid, rifampicin, beta-lactam antibiotics, penicillin, amoxicillin, doxycycline, cephalexin, ciprofloxacin, clindamycin, metronidazole, azithromycin, sulfamethoxazole, trimethoprim, Ampicillin, Penicillin G, Penicillin V, cephalosporins, Cefixime, Cefpodoxime, Cefuroxime, Cephalexin, macrolides, Azithromycin (‘Z-pak’), Clarithromycin, Erythromycin, Fluoroquinolones (or quinolones), Ciprofloxacin, Levofloxacin, Moxifloxacin, sulfonamides, Sulfacetamide, Sulfadiazine, Sulfamethoxazole-Trimethoprim, tetracycline, Doxycycline, Minocycline, Tetracycline, clindamycin, metronidazole and/or nitrofurantoin. Antimicrobials such as broad-spectrum antibiotics (one or more β-lactam antibiotic with broad coverage, antibiotics specific to gram-negative organisms, or broad-spectrum carbapenem combined with fluoroquinolones, macrolides, or aminoglycosides. The choice of antibiotics is important in determining the course of treatment and survival of a subject/patient.

### Kits

The traditional sepsis diagnosis workflow requires pathogen cultivation, which increases time to diagnosis. Additionally, this approach often results in false negatives, which, as recently demonstrated by the COVID-19 crisis, pose a serious public health threat. Newer tools can generally be classified into three categories: 1) preliminary diagnostic for sepsis. This could be used in conjunction with technology discussed herein but does not inform physicians of proper antibiotic treatment; 2) tool to identify the pathogen. This can only loosely advise as to which antibiotic should be used but does not account for resistant strains, which will become a wider gap as the antibiotic resistance crisis continues to expand; and 3) tool that reports antibiotic susceptibility using known mutations as a proxy for resistance. However, the limited number of mutations tested cannot account for all resistance outcomes. This restriction will inevitably also become a graver problem with the rise of antibiotic resistance.

Provided herein is a sepsis diagnostic kit, such as a sepsis diagnostic kit, that enables clinical laboratories to reduce the time to an antibiotic susceptibility profile. This improvement 1) increases patient survival, saving as many as 900,000 patients per year in the U.S., 2) reduces healthcare costs by up to $8,000 per sepsis patient due to shorter hospital stays and more effective, personalized treatment, and 3) improves quality of patient care and hospital rankings, which are driving forces in healthcare decision making. This kit uses otherwise-undetectable cell growth as a marker for antibiotic susceptibility, yielding a combination of features that is currently unmet by other diagnostic technologies: rapid, accurate, sensitive, cheap, and relies on standard clinical laboratory equipment.

Provided herein is a kit for rapid drug susceptibility profiling of blood-borne pathogenic bacteria. There are other products that predict antibiotic susceptibility rapidly. However, these technologies either involve specialized, expensive equipment, or require a high bacterial load, which prolongs time to diagnosis.

The provides reagents, including modified and optionally labeled D-Amino acids and/or trehalose, as well as instructions on how to use the kit.

The following examples are intended to further illustrate certain embodiments of the invention and are not intended to limit the scope of the invention in any way.

### EXAMPLES

### Example I: Guidance of Sepsis Treatment by Bacterial Cell Envelope Labeling/Diagnostic for Detection and Antibiotic Susceptibility Profiling of Bacterial Pathogens

### INTRODUCTION

Bloodstream bacterial infections are estimated to impact 1-3 million U.S. patients each year and are a major cause of death for soldiers injured in combat (1, 2). The in-hospital mortality rate for severe sepsis is 15-30% (1). Currently, it takes approximately 24 hours to obtain a positive blood culture, approximately 24 hours to identify the bacterial species, and approximately 18-24 hours for antibiotic susceptibility testing, for a total 3-day turnaround. The only known correlate of sepsis survival is the time elapsed prior to initiation of correct antibiotic treatment (3-5). Shortening the time to (proper)diagnosis can dramatically improve patient outcomes, reduce hospital costs and promote antimicrobial stewardship (3-9).

The primary bottleneck in the diagnosis of bloodstream bacterial infections is the length of time it takes for the organisms to replicate to a detectable density. Provided herein is metabolic labeling of the bacterial cell surface that provides a rapid, amplifiable readout that 1) will shorten the time to initial confirmation of bloodstream bacterial infection and subsequent determination of antibiotic susceptibility 2) does not require speciation.

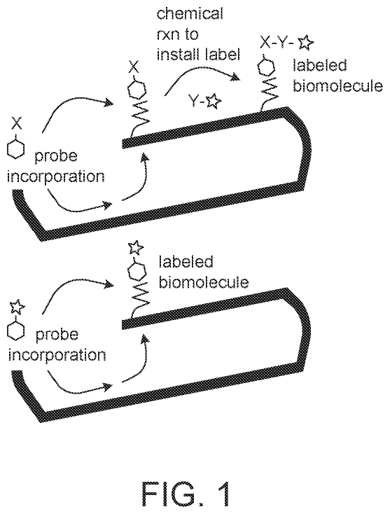

The cell surface is a conserved feature of the bacterial cell. Probes that mimic envelope precursors metabolically incorporate into the cell surface within minutes and can be revealed by numerous detection moieties including fluorophores or affinity handles (FIG. 1, (10-12)). The data provided herein support the belief that new envelope is a marker for bacterial replication. Further, antibiotic-resistant bacteria will grow in the presence of drug (envelope-acting or not) and be marked by precursor probes whereas antibiotic-susceptible bacteria will not.

### Materials and Methods

Bacterial strains and growth conditions. Staphylococcus aureus was grown in brain heart infusion broth (BHI; BD BBL, Sparks, MD.). Escherichia coli K-12 was grown in Luria Bertani broth (LB; Apex Research Products, El Cajon, CA). All bacteria were initially grown by shaking at 37° C. overnight.

Cell wall labeling. Bacterial cultures were back diluted in their respective media and grown at 37° C. until the mid-log phase (OD₆₀₀=0.3 for S. aureus; OD₆₀₀=0.35 for E. coli); for concentration dependency experiments the starting OD₆₀₀ for each sample varied. Where appropriate, cultures were treated with freshly prepared ampicillin (100 μg/mL; Fisher Bioreagents, Fair Lawn, NJ) or vancomycin (100 μg/mL; Alfa Aesar, Ward Hill, MA) and incubated on a shaker at 37° C. for 90 minutes.

Growing bacteria were labeled with biotin D-lysine (Chem-Impex International, Wood Dale, IL; reagent was deprotected first). Unless otherwise indicated, they were labeled with 1 mM biotin-D-lysine shaking at 37° C. for 20 (S. aureus) or 30 (E. coli) minutes. Bacteria were then pelleted by centrifugation (5000×g for 5 minutes) and washed in cold PBS three times. In the case of S. aureus, cells were additionally fixed for 20 minutes in ice with 4% paraformaldehyde and further washed three times in PBS.

ELISA preparation. Uncoated ELISA plates (ThermoFisher Scientific, Rochester, NY) were coated using 100 μl of 10 μg/ml streptavidin (New England Biolabs, Rowley, MA) in carbonate coating buffer (8.4 g NaHCO₃, 3.56 g Na₂CO₃, add ddH₂O up to 1 L; pH to 9.5) and incubated at 4° C. overnight. The following day, plates were washed three times with wash buffer (0.05% (v/v) Tween®-20 (Amresco, Solon, OH) in PBS). The plates were then incubated with 200 μl of freshly made and filtered blocking solution (1% BSA in 0.05% (v/v) Tween®-20 in PBS) per well at 4° C. overnight.

ELISA experiment. Blocked ELISA plates were washed three times using the wash buffer (described above). Cells resuspended in freshly made and filtered blocking solution (described above) were added to the plates in triplicate and incubated for 90 minutes at room temperature or at 4° C. overnight. Afterwards, the plates were washed three times and incubated with 100 μl streptavidin-HRP (diluted 1:10,000; ThermoFisher Scientific, Waltham, MA) for 30 minutes at room temperature. After incubation, the plates were washed three times and 100 μl of freshly prepared substrate solution (BD OptEIA, San Diego, CA) was added to each well. The reaction was stopped after 30 minutes using 100 μl of stop solution (2 M sulfuric acid) per well. The absorbance was measured at 450 nm with a BioTek Plate Reader.

### Results

Chemical probes were synthesized for tagging cell surface components of pathogenic bacteria (FIG. 1, (10, 11, 20-22)). Recently, the cell wall incorporation pathway(s) for a panel of cell envelope precursors in, for example mycobacterium, was determined (16).

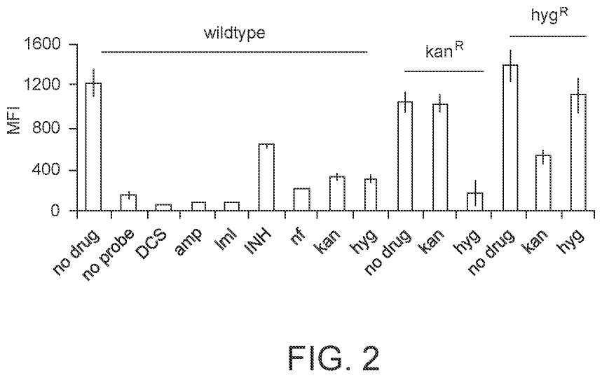

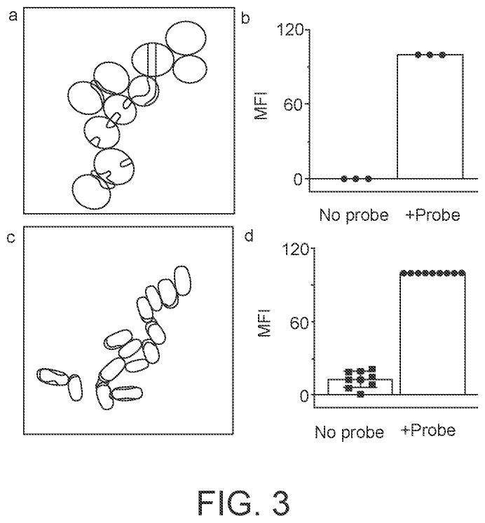

Provided herein are aspects that bacterial, such as mycobacterial, surface labeling is sensitive to growth inhibition by antibiotics with diverse mechanisms of action (FIG. 2). The invention can be used with all bacteria, including pathogenic bacteria. As shown herein, the cell surface probes incorporate into diverse species, including E. coli and S. aureus (FIG. 3, (10-12)), respectively the most common Gram-negative and Gram-positive causes of bacteremia (13-15).

In one aspect, to make this labeling technique a clinically relevant diagnostic tool, an ELISA protocol was developed. The experiments with S. aureus demonstrate that this method is a specific, and rapid approach to test antibiotic susceptibility (FIG. 4; ELISA readout of the growth-dependent metabolic labeling).

### Discussion

Provided herein is an ELISA readout which demonstrates that it is dose and time-dependent and can detect metabolic labeling of both Staphylococcus aureus and Escherichia coli, respectively the most common Gram-positive and Gram-negative causes of bacterial sepsis. Further, the assay can discriminate between S. aureus+/−vancomycin, the antibiotic of choice for methicillin-resistant S. aureus (MRSA) infections.

The data was generated by metabolic labeling of sepsis-relevant bacteria using D-amino acid derivatives. However, this could also be accomplished using other derivatives, such as trehalose derivatives (doi: 10.1021/ja3062419) in mycobacteria (which can be used to diagnose tuberculosis, as well as non-tuberculosis mycobacterial infections).

### BIBLIOGRAPHY

1. Gaieski D F, Edwards J M. Kallan M J, Carr B G. 2013. Benchmarking the incidence and mortality of severe sepsis in the United States. Critical Care Medicine 41:1167-1174.
2. Johnston A M. Easby D, Ewington I. 2013. Sepsis management in the deployed field hospital. Journal of the Royal Army Medical Corps 159:175-180.
3. Doern G V, Vautour R, Gaudet M. Levy B. 1994. Clinical impact of rapid in vitro susceptibility testing and bacterial identification. Journal of Clinical Microbiology 32:1757-1762.
4. Garnacho-Montero J, Aldabo-Pallas T, Garnacho-Montero C, Cayuela A, Jimenez R, Barroso S, Ortiz-Leyba C. 2006. Timing of adequate antibiotic therapy is a greater determinant of outcome than are TNF and IL-10 polymorphisms in patients with sepsis. Critical Care 10: R111.
5. Barenfanger J, Drake C, Kacich G. 1999. Clinical and financial benefits of rapid bacterial identification and antimicrobial susceptibility testing. Journal of Clinical Microbiology 37:1415-1418.
6. Biendo M. Mammeri H, Pluquet E, Guillon H, Rousseau F, Canarelli B, Belmekki M. Eb F. 2013. Value of Xpert MRSA/SA blood culture assay on the Gene Xpert® Dx System for rapid detection of Staphylococcus aureus and coagulase-negative staphylococci in patients with staphylococcal bacteremia. Diagnostic Microbiology and Infectious Disease 75:139-143.
7. Blaschke A J, Heyrend C, Byington C L, Fisher M A, Barker E, Garrone N F, Thatcher S A, Pavia A T, Barney T, Alger G D, Daly J A, Ririe K M. Ota I, Poritz M A. 2012. Rapid identification of pathogens from positive blood cultures by multiplex polymerase chain reaction using the FilmArray system. Diagnostic Microbiology and Infectious Disease 74:349-355.
8. Galar A, Leiva J, Espinosa M. Guillen-Grima F, Hernaez S, Yuste J R. 2012. Clinical and economic evaluation of the impact of rapid microbiological diagnostic testing. The Journal of Infection 65:302-309.
9. Galar A, Yuste J R, Espinosa M. Guillen-Grima F, Hernaez-Crespo S, Leiva J. 2012. Clinical and economic impact of rapid reporting of bacterial identification and antimicrobial susceptibility results of the most frequently processed specimen types. European Journal of Clinical Microbiology & Infectious Diseases 31:2445-2452.
10. Siegrist M S, Swarts B M. Fox D M. Lim S A, Bertozzi C R. 2015. Illumination of growth, division and secretion by metabolic labeling of the bacterial cell surface. FEMS Microbiology Reviews 39:184-202.
11. Siegrist M S, Whiteside S, Jewett J C, Aditham A, Cava F, Bertozzi C R. 2013. D-amino acid chemical reporters reveal peptidoglycan dynamics of an intracellular pathogen. ACS Chemical Biology 8:500-505.
12. Kuru E, Hughes H V, Brown P J, Hall E, Tekkam S, Cava F, de Pedro M A, Brun Y V, VanNieuwenhze M S. 2012. In Situ probing of newly synthesized peptidoglycan in live bacteria with fluorescent D-amino acids. Angewandte Chemie 51:12519-12523.
13. Antonio M. Gudiol C, Royo-Cebrecos C, Grillo S, Ardanuy C, Carratala J. 2019. Current etiology, clinical features and outcomes of bacteremia in older patients with solid tumors. Journal of Geriatric Oncology 10:246-251.
14. Munro A P S, Blyth C C, Campbell A J, Bowen A C. 2018. Infection characteristics and treatment of Staphylococcus aureus bacteraemia at a tertiary children's hospital. BMC Infectious Diseases 18:387.
15. Woll C, Neuman M I, Pruitt C M. Wang M E, Shapiro E D, Shah S S, McCulloh R J, Nigrovic L E, Desai S, DePorre A G, Leazer R C, Marble R D, Balamuth F, Feldman E A, Sartori L F, Browning W L, Aronson P L, Febrile Young Infant Research C. 2018. Epidemiology and Etiology of Invasive Bacterial Infection in Infants </=60 Days Old Treated in Emergency Departments. The Journal of Pediatrics 200:210-217 e211.
16. Garcia-Heredia A, Pohane A A, Melzer E S, Carr C R, Fiolek T J, Rundell S R, Chuin Lim H, Wagner J C, Morita Y S, Swarts B M. Siegrist M S. 2018. Peptidoglycan precursor synthesis along the sidewall of pole-growing mycobacteria. eLife 7.
17. Melzer E S, Sein C. E., Chambers J. J., Siegrist M. S. 2018. DivIVA concentrates mycobacterial cell envelope assembly for initiation and stabilization of polar growth. Cytoskeleton.
18. Hsu Y P, Rittichier J, Kuru E, Yablonowski J, Pasciak E, Tekkam S, Hall E, Murphy B, Lee T K, Garner E C, Huang K C, Brun Y V, VanNieuwenhze M S. 2017. Full color palette of fluorescent D-amino acids for in situ labeling of bacterial cell walls. Chemical Science 8:6313-6321.
19. Kuru E, Tekkam S, Hall E, Brun Y V, Van Nieuwenhze M S. 2015. Synthesis of fluorescent D-amino acids and their use for probing peptidoglycan synthesis and bacterial growth in situ. Nature protocols10: 33-52.
20. Shieh P, Siegrist M S, Cullen A J, Bertozzi C R. 2014. Imaging bacterial peptidoglycan with near-infrared fluorogenic azide probes. Proceedings of the National Academy of Sciences of the United States of America 111:5456-5461.
21. Siegrist M S, Aditham A K, Espaillat A, Cameron T A, Whiteside S A, Cava F, Portnoy DA, Bertozzi CR. 2015. Host actin polymerization tunes the cell division cycle of an intracellular pathogen. Cell Reports 11:499-507.
22. Swarts B M. Holsclaw C M. Jewett J C, Alber M. Fox D M. Siegrist M S, Leary J A, Kalscheuer R, Bertozzi C R. 2012. Probing the mycobacterial trehalome with bioorthogonal chemistry. Journal of the American Chemical Society 134:16123-16126.

Siegrist M S, Jewett J C, Shieh P, Gordon C G, Bertozzi C R. 2016. “D-amino acid derivative-modified peptidoglycan and methods of use thereof.” U.S. Pat. No. 9,303,068

### Example II

Provided herein is an ELISA to detect metabolic labeling of the mycobacterial cell envelope. Actively growing, for example, Mycobacterium smegmatis incorporates biotinylated D-amino acid and trehalose derivatives into their cell wall peptidoglycan and mycomembrane, respectively. One can then detect the labeled bacteria by, for example, using a sandwich ELISA to first capture labeled M. smegmatis then amplify the signal.

Metabolic labeling of cell macromolecules is generally detected by radioactivity, fluorescence and/or immunoblot. While these methods work well in a research laboratory setting, they are generally not well-suited to clinical applications. As described herein, an ELISA was developed for use as a diagnostic. The data suggest that it is a rapid, amplifiable readout for active growth of bacteria, such as Staphylococcus aureus and Escherichia coli, the most common Gram-positive and Gram-negative culprits of bacterial sepsis. This was also demonstrated with Mycobacterium smegmatis, an organism that is phylogenetically related to Gram-positives but has an outer ‘myco’ membrane that renders it Gram-indeterminate. It was also show that one can use an ELISA to detect labeling via trehalose, a mycobacterial-specific metabolite. While the D-amino acid labeling method will enable detection of pathogenic mycobacteria from the blood, an otherwise-sterile environment, trehalose labeling will enable detection from samples and tissues in which there are other many other bacterial species, e.g., sputum. This has significant use for as a diagnostic for detection and antibiotic susceptibility profiling of mycobacterial pathogens in clinical setting, as well as detecting bacterial growth in the food industry and manufacturing sector.

The assay used disclosed herein would not need to make use of a fluorophore. By pairing detection with metabolic labeling, the method can detect very low levels of bacteria, for example with a lower detection limit of in the order of about 10⁴ CFU/mL or lower (removing or minimizing outgrowth/culturing the bacteria prior to testing for bacteria/diagnosing), which allows for quick processing times (shortening the clinical diagnosis/process by days; (or example, metabolic labeling of about 1.5 hours and detection of about 3 hours)) even for samples with very few bacteria, such as 11-10⁴ CFU/ml. For example, one could label a D-amino acid (such as D-alanine) with a biotin followed by labeling with a reporter. For example, the reporter could be an enzyme that converts the color of a substrate molecule. Once metabolically labeled with D-amino acid, the bacteria could then be captured by a biotin-binding protein, such as streptavidin, avidin or deglycosylated avidin coated on a plate. This would allow one to amplify the metabolic labeling for detection of bacteria by using, for an example, an ELISA (a benefit of using an avidin-biotin system for detection is the ability to amplify the original signal to improve detection of low levels of analytes by forming large avidin-biotin complexes. These complexes enrich the conjugated reporter (fluorophore or enzyme or antibody) to the site of the target antigen for greater signal detection).

All publications, patents and patent applications are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

1. A method to determine if an antibiotic is effective to treat a bacterial infection in a subject comprising:
a) contacting a sample from a subject suffering from a bacterial infection with an antibiotic; and
b) determining the effect, if any, of the antibiotic on incorporation of a single- or di-D-amino acid into peptidoglycan of the bacterial cell or incorporation of trehalose into the bacterial cell envelope,
wherein an antibiotic that reduces, as compared to control without the antibiotic being present, incorporation of the D-amino acid into peptidoglycan of the bacterial cell or inhibited incorporation of trehalose into the bacterial cell envelope is an antibiotic that is effective to treat said bacterial infection in said subject.

2. A method to treat a subject for a bacterial infection comprising:
a) contacting a sample from a subject suffering from a bacterial infection with an antibiotic;
b) determining the effect of the antibiotic on incorporation of a single or di-D-amino acid into peptidoglycan of the bacterial cell or trehalose incorporation into the bacterial cell envelope; and
c) administering the antibiotic to said subject, wherein inhibited incorporation of the D-amino acid into peptidoglycan of the bacterial cell or inhibited incorporation of trehalose into the bacterial cell envelope was determined in b), so as to treat said subject for a bacterial infection.

3. A method to detect antibiotic resistant strains of bacteria in a subject comprising:
a) contacting a sample from a subject suffering from a bacterial infection with an antibiotic; and
b) determining the effect, if any, of the antibiotic on incorporation of a single- or di-D-amino acid into peptidoglycan of the bacterial cell or trehalose incorporation into the bacterial cell envelope,
wherein the bacteria are resistant to said antibiotic if said bacteria continue to grow and incorporate the D-amino acid into peptidoglycan of the bacterial cell or trehalose incorporation into the bacterial cell envelope.

4. A method to detect bacterial contamination of a site comprising,
a) contacting a sample from a site with an antibiotic; and
b) determining the effect, if any, of the antibiotic on incorporation of a single- or di-D-amino acid into peptidoglycan of the bacterial cell or trehalose incorporation into the bacterial cell envelope,
wherein an antibiotic that reduces, as compared to control without the antibiotic being present, incorporation of the D-amino acid into peptidoglycan in the bacterial cell or inhibited incorporation of trehalose is indicative of bacterial contamination of said site.

5. The method of claim 2, wherein the sample comprises cells and bacteria or the method of claim 4 wherein the sample is in the form of a liquid obtained from the site or swab or other wiping of said site.

6. The method of claim 2, wherein the bacterial infection or contamination is caused by a pathogenic bacterium.

7. The method of claim 6, wherein said pathogenic bacterium is one or more of the bacteria that contribute to sepsis.

8. The method of claim 6, wherein said bacterium is a mycobacterium.

9. The method of claim 2, wherein the D-amino acid is modified with a bioorthogonal functional group.

10. The method of claim 9, wherein the bioorthogonal functional group is an azide, an alkyne, or a norbornene group.

11. The method of claim 2, wherein the D-amino acid and/or trehalose is labeled with a detectable label.

12. The method of claim 11, wherein the detectable label is biotin, a radioactive isotope, a fluorescent label or a peptide that can be detected by antibody binding.

13. The method of claim 2, wherein the identity of the bacteria causing the infection or contamination is not determined.

14. The method of claim 2, wherein b) is carried out by an ELISA assay.

15. The method of claim 4, wherein the site is an industrial setting and/or hospital facility setting (buildings, rooms, beds, beds, chairs etc.), including a food production facility (factory or bakery), biomedical reagent or device facility, non-food products facility and products, such as a home care products (cleaning products), personal care products (cosmetics (skin care, hair care, make up, baby care products; and toiletries (toothcare, feminine care products)), chemical products and toys, including, but not limited to, tattoo ink and children's toys such as soap bubbles, play dough and paints, as well water for drinking or other uses.

16. The method of claim 4, wherein the contaminated site is decontaminated and optionally progress of said decontamination is monitored by repeating steps a) and b) one or more times.

17. A method to detect low amounts of bacteria in a subject or site comprising:
a) metabolically labeling bacteria in a sample from a subject or site by incorporation of a single- or di-D-amino acid into peptidoglycan of the bacterial cell, wherein the D-amino acid is labeled with biotin;
b) capturing said metabolically labeled bacteria with avidin; and
c) detecting the signal with an ELISA, IHC, or western blot,
wherein the bacteria are grown out/cultured/expanded prior to and/or during metabolic labeling.

18. A method to detect low amounts of bacteria in a subject or site comprising:
a) metabolically labeling bacteria in a sample from a subject or site by incorporation of a single- or di-D-amino acid into peptidoglycan of the bacterial cell;
b) capturing said metabolically labeled bacteria with an antibody; and
c) detecting said antibody,
wherein the bacteria are grown out/cultured/expanded prior to and/or during metabolic labeling.

19. The method of claim 18, wherein b) and c) are carried out by a lateral flow assay.
